(19) United States
(12) Patent Application Publication
(10) Pub. No.: US 2004/0002601 A1
(43) Pub. Date: Jan. 1, 2004

(54) Method for producing cephalosporins

(75) Inventors: Maurizio Zenoni, Paullo (IT); Giovanni Fogliato, Barzana (IT); Mauro Filippi, Bettolino Di Mediglia (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: ACS DOBFAR S.p.A., Tribiano (IT)

(21) Appl. No.: 10/347,459

(22) Filed: Jan. 21, 2003

(30) Foreign Application Priority Data
Jun. 28, 2002 (IT) MI2002A 001432

(51) Int. Cl. C07D 501/14; C07F 9/6539

(52) U.S. Cl. 540/228; 548/112

(57) ABSTRACT

A method for producing cephalosporins 7-substituted with an amino-thiazolylacetic group by reacting 7-ACA or its derivatives having the amino group and the carboxyl protected with reactive derivatives of amino-thiazolylacetic acid.

### FIELD OF THE INVENTION

The present invention relates to a method for producing cephalosporins 7-substituted with an amino-thiazolylacetic group of formula

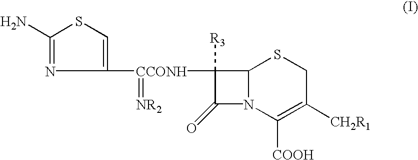

in which R₁ is hydrogen or a residue of a nucleophilic compound, R₂ is a hydroxyl or substituted hydroxyl, R₃ is hydrogen or methoxyl.

U.S. Pat. No. 5,567,813 describes a method for producing cephalosporins of formula (I)—in which however R₃ is only hydrogen—according to which an acyl group of formula

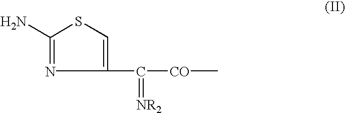

is introduced into the amino group of molecules of formula

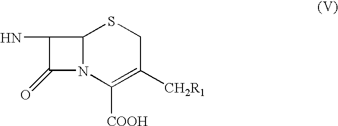

by reacting a compound of formula (V) with a compound of formula

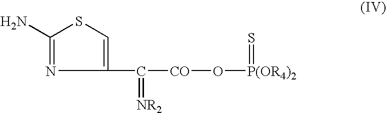

where R₄ is a C₁-C₄ alkyl.

### BACKGROUND OF THE INVENTION

The alkyl groups (II) and their preparation are described in U.S. Pat. No. 4,152,432 and U.S. Pat. No. 4,327,210; the preparation of the compounds of formula (IV) is described in U.S. Pat. No. 5,502,200 in the name of the same proprietor as U.S. Pat. No. 5,567,813.

### DISCUSSION OF THE RELATED ART

According to U.S. Pat. No. 5,567,813, the compound (IV) is reacted with the compound (V) in which the reactive groups NH₂ and COOH are free: it has been noted that for certain meanings of R₁, the method described in the US patent leads to the production of cephalosporins (I) with high yields and purities, whereas for other meanings of R₁, to give certain important cephalosporins such as cefpirome (Examples 13 and 14) and cefepime (Examples 15 and 16), the yields are decidedly lower.

### BRIEF SUMMARY OF THE INVENTION

The object of the present invention is to provide a process which can be easily implemented to produce all cephalosporins of formula (I) with the same ease and with high yields.

### DETAILED DESCRIPTION OF THE INVENTION

This process is characterised by introducing an acyl group of formula

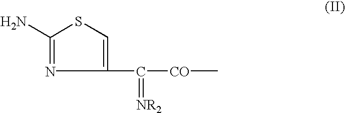

into the amino group of a molecule of formula

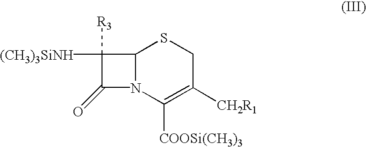

by reacting in an anhydrous organic solvent a compound of formula (III) with a compound of formula

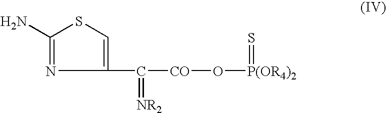

where R₄ is a C₁-C₄ alkyl, finally removing the trimethylsilyl groups by known methods, to give the cephalosporins of formula (I).

Preferably said residue of a nucleophilic compound is chosen from the group consisting of methoxy, acetoxy, (1-methyl-1H-tetrazol-5-yl)thio, (5-carboxymethyl-4-methyl-2-thiazolyl)thio, (2-furanylcarbonyl) thio, (2,5-dihydro-6-hydroxy-2 -methyl-5-oxo-as-triazin-3-yl)thio, 1-methylpyrrolidine, 2,3-cyclopentene-1-pyridine, 1-(5,6,7,8-tetra-hydroquinoline) and 1-pyridine, said substituted hydroxyl being chosen from the group consisting of methoxyl and 1-carboxy-1-methylethoxy.

Again preferably, said inert anhydrous organic solvent is chosen from the group consisting of methylene chloride, ethylacetate and DMF.

The compounds of formula (III) are easily obtained from molecules of formula (V) in the manner described in detail in EP-B-0612750. Some embodiments of the invention will now be described in detail.

### EXAMPLE 1

10 g of 7-amino-3-(2,5-dihydro-2-methyl-6-hydroxy-5-oxo-as-triazin-3-yl)thiomethyl-3-cephem-4-carboxylic acid are suspended in 70 ml of dichloromethane. 18.4 g of N,O-bis-(trimethylsilyl)acetamide are added and the mixture is agitated for 2 h at ambient temperature.

16.2 g of diethylthiophosphoryl-(Z)-(2-aminothiazol-4-yl)methoxyimino acetate are added and the mixture left to react at ambient temperature for 4 hours. 30 ml of water are added to the reaction mixture and 30% NaOH is dripped in at 15°/20° C. until pH 7.5-7.8 is attained.

The aqueous phase is separated and is treated for 20 minutes at ambient temperature with 1 g of carbon, then filtered and washed with 15 ml of water. 60 ml of acetone and 3 2 g of NaCl are added. Crystallization commences and the mixture is left under agitation for 1 h.

240 ml of acetone are finally dripped in to complete the precipitation. The mixture is filtered, washed with 20 ml of 9:1 acetone/water and then with 400 ml of acetone.

It is dried at +40° C. under reduced pressure.

16.8 g of ceftriaxone disodium hemiheptahydrate are obtained (titre 84% as anhydrous acid). Molar yield: 94.6%.

Ceftriaxone is a compound of formula (I) in which R₁ is (2,5-dihydro-6-hydroxy-2-methyl-5-oxo-as-triazin-3-yl)thio, R₂ is methoxyl and R₃ is hydrogen.

### EXAMPLE 2

10 g of 7-amino-3-(2,5-dihydro-2-methyl-6-hydroxy-5-oxo-as-triazin-3-yl)thiomethyl-3-cephem-4-carboxylic acid are suspended in 50 ml of anhydrous DMF. 18.4 g of N,O-bis-(trimethylsilyl)acetamide are added and the mixture is agitated for 2 h at ambient temperature.

16.2 g of diethylthiophosphoryl-(Z)-(2-aminothiazol-4-yl)methoxyimino acetate are added and the mixture left to react at ambient temperature for 4 hours. 30 ml of water are added to the reaction mixture and 30% NaOH is dripped in at +15/+20° C. until pH 7.5-7.8 is attained.

The aqueous solution is washed repeatedly with dichloromethane.

The resultant aqueous solution is treated for 20 minutes at ambient temperature with 1 g of carbon, then filtered and washed with 15 ml of water. 250 ml of acetone are rapidly added and the mixture is left under agitation to crystallize for 1 h.

The mixture is filtered, washed with 20 ml of 9:1 acetone/water and then with 400 ml of acetone.

It is dried at +40° C. under reduced pressure.

16 g of ceftriaxone disodium hemiheptahydrate are obtained (titre 82% as anhydrous acid).

### EXAMPLE 3

10 g of 7-amino-3-(2,5-dihydro-2-methyl-6-hydroxy-5-oxo-as-triazin-3-yl)thiomethyl-3-cephem-4-carboxylic acid are suspended in 70 ml of dichloromethane. 5.9 g of trimethylchlorosilane and 8.7 g of hexamethyldisilazane are added. The mixture is agitated for 2 h at ambient temperature.

16.2 g of diethylthiophosphoryl-(Z)-(2-aminothiazol-4-yl)methoxyimino acetate are added and the mixture left to react at ambient temperature for 18 hours.

Operating as in Example 1 results are obtained which are quantitatively perfectly superimposable.

### EXAMPLE 4

10 g of 7-amino-3-(2,5-dihydro-2-methyl-6-hydroxy-5-oxo-as-triazin-3-yl)thiomethyl-3-cephem-4-carboxylic acid are suspended in 70 ml of dichloromethane. 16.7 g of N,N′-bis-trimethyl-silylurea are added and the mixture is agitated for 2 h at ambient temperature.

16.2 g of diethylthiophosphoryl-(Z)-(2-aminothiazol-4-yl)methoxyimino acetate are added and the mixture left to react at ambient temperature for about 10 hours.

30 ml of water are added to the reaction mixture and 30% NaOH is dripped in at +15°/20° C. until pH 7.5-7.8 is attained.

The aqueous phase is separated and is treated for 20 minutes at ambient temperature with 1 g of carbon, then filtered and washed with 15 ml of water. 60 ml of acetone are added. Crystallization commences and the mixture is left under agitation for 1 h.

240 ml of acetone are finally dripped in to complete the crystallization. The mixture is filtered, washed with 20 ml of 9:1 acetone/water and then with 400 ml of acetone.

It is dried at +40° C. under reduced pressure.

16.7 g of ceftriaxone disodium hemiheptahydrate are obtained (titre 83% as anhydrous acid).

### EXAMPLE 5

10 g of 7-aminocephalosporanic acid are suspended in 70 ml of dichloromethane. 7.9 g of N,O-bis-(trimethylsilyl)acetamide are added and the mixture is agitated for 1 h at ambient temperature.

8.1 g of diethylthiophosphoryl-(Z)-(2-aminothiazol-4-yl)methoxyimino acetate are added and the mixture left to react at ambient temperature for 8 hours.

On termination of the reaction the synthesis mixture is dripped into 80 ml of water at +15/+20° C., adjusting the pH to 7.5-7.8 with 15% NaOH during the dripping.

The aqueous phase is separated, diluted with 16 ml of isopropyl alcohol and then with water to a total of 195 ml. 5 ml of ethyl acetate are added to the solution obtained, it is cooled to 0° C. and 15% HCl added until pH 3.5 is achieved, where the first crystals appear. The mixture is agitated for 30 min and the pH then lowered to 2.7. It is again agitated for 30 min and filtered, washing with acetone.

It is dried at +40° C. under reduced pressure.

8.5 g of cefotaxime acid are obtained.

Molar yield: 94.4%.

Cefotaxime is a compound of formula (I) in which R₁ is acetoxy, R₂ is methoxyl and R₃ is hydrogen.

### EXAMPLE 6

5.0 g of 7-aminocephalosporanic acid are suspended in 35 ml of anhydrous DMF.

7.3 g of N,O-bis-(trimethylsilyl)acetamide are added while maintaining the temperature at +20°/+25° C. The 7-aminocephalosporanic acid dissolves rapidly and totally.

7.5 g of diethylthiophosphoryl-(Z)-(2-aminothiazol-4-yl)methoxyimino acetate are added and the mixture left to react at ambient temperature for 18 hours. On termination of the reaction 65 ml of water and 65 ml of methylene chloride are added.

The mixture is adjusted to pH 7 with NaHCO₃ and the phases are separated. The aqueous phase is washed repeatedly with dichloromethane.

6 ml of isopropyl alcohol are added to the aqueous phase and then diluted to a total of 195 ml with water. 5 ml of ethyl acetate are added to the solution obtained, it is cooled to 0° C. and 15% HCl added until pH 3.5 is achieved, where the first crystals appear. The mixture is agitated for 30 min and the pH then lowered to 2.7.

It is again agitated for 30 min and filtered, washing with acetone.

It is dried at +40° C. under reduced pressure.

7.7 g of cefotaxime acid are obtained.

Molar yield: 93.5%.

### EXAMPLE 7

10 g of 7-amino-3-[(1-methyl-1-pyrrolidine)methyl]-3-cephem-4-hydroiodide are added to 300 ml of dichloromethane in a nitrogen atmosphere. Trimethylchlorosilane (4.7 ml) and hexamethyl disilazane (7.7 ml) are added, the temperature is adjusted to 25°/30° C. and the mixture is agitated for 2 hours, again at 25°/30° C. The mixture is cooled to 20° C., 11.5 g of diethylthiophosphoryl-(Z)-(2-aminothiazol-4-yl)methoxyimino acetate are added, and the mixture left under agitation at 20°/+25° C. overnight. The reaction mixture is added slowly to water (50 ml) and after 60 minutes of agitation the phases are separated. The aqueous phase is washed with dichloromethane and 6N hydrochloric acid and acetone (100 ml) are added to the aqueous phase. The mixture is left to crystallize for 30 minutes and further acetone (180 ml) are then added to complete the crystallization. After 60 minutes of agitation the mixture is filtered, washed with acetone and dried at 40° C. 12.2 g of cefepime dihydrochloride monohydrate are obtained.

Molar yield: 94.8%.

### EXAMPLE 8

5 g of 7-amino-3-(2-furanylcarbonyl)thiomethyl-3-cephem-4-carboxylic acid are suspended in 35 ml of dichloromethane. 5.5 g of N,O-bis-(trimethylsilyl)acetamide are added and the mixture is agitated for 3 h at ambient temperature.

6.4 g of diethylthiophosphoryl-(Z)-(2-aminothiazol-4-yl)methoxyimino acetate are added and the mixture left to react at ambient temperature for 6 hours.

50 ml of water are added to the solution at the end of the reaction and 30% NaOH is dripped in until pH 7.5 is attained. The aqueous phase is separated and decolorized with 1 g of carbon for 20 min.

After filtration 50 ml of 36% HCl are slowly dripped in until pH 3 is attained. The organic phase is separated, 3.5 g of sodium 2-ethylhexanoate are added and the mixture left to crystallize at 0° C. for 8 h, then filtered, washing with cold tetrahydrofuran.

It is dried at +30° C. under reduced pressure.

9.2 g of ceftiofur sodium salt are obtained.

Molar yield: 90%

Proceeding in the same manner as the aforedescribed Examples, but using reagents of formula (III) in which R₁ is chosen from the group consisting of methoxy, (1-methyl-1H-tetrazol-yl)thio, (5-carboxymethyl-4-methyl-2-thiazolyl)thio, 2, 3-cyclopentene-1-pyridine, 1-(5,6,7,8-tetrahydro-quinoline) and 1-pyridine, and R₂ is chosen from the group consisting of methoxy and 1-carboxy-1-methylethoxy, other important cephalosporins are obtained, known by the name of cefmenoxime, cefodizime, cefpirome, cefpodoxime (from which cefpodoxime proxethyl can be prepared), cefquinome and ceftazidime.

What we claim is:

1. A method for producing cephalosporins 7-substituted with an amino-thiazolylacetic group of formula 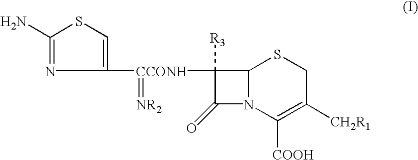
in which R₁ is hydrogen or a residue of a nucleophilic compound, R₂ is a hydroxyl or substituted hydroxyl, R₃ is hydrogen or methoxyl, wherein an acyl group of formula 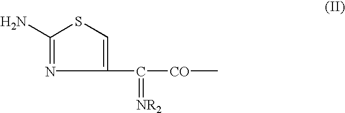
is introduced into the amino group of a molecule of formula 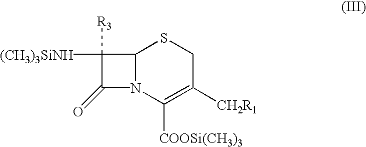
by reacting in an anhydrous organic solvent a compound of formula (III) with a compound of formula 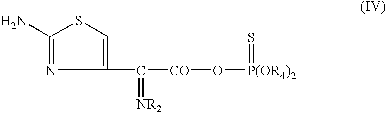
where R₄ is a C₁-C₄ alkyl, finally removing the trimethylsilyl groups by known methods, to give the cephalosporins of formula (I).

2. A method as claimed in claim 1, wherein said residue of a nucleophilic compound is chosen from the group consisting of methoxy, acetoxy, (1-methyl-1H-tetrazol-5-yl)thio, (5-carboxymethyl-4-methyl-2-thiazolyl)thio, (2-furanylcarbonyl)thio, (2,5-dihydro-6-hydroxy-2-methyl-5-oxo-as-triazin-3-yl)thio, 1-methyl-pyrrolidine, 2,3-cyclopentene-1-pyridine, 1-(5,6,7,8-tetrahydro-quinoline) and 1-pyridine, said substituted hydroxyl being chosen from the group consisting of methoxy and 1-carboxy-1-methylethoxy.

3. A method as claimed in claims 1 wherein said inert anhydrous organic solvent is chosen from the group consisting of methylene chloride, ethylacetate and DMF.

4. A method as cliamed in claim 2 wherein said inert anhydrous organic solvent is chosen from the group consisting of methylene chloride, ethylacetate and DMF.
